(19) United States
(12) Patent Application Publication
(10) Pub. No.: US 2016/0041141 A1
(43) Pub. Date: Feb. 11, 2016

(54) METHOD FOR SORTING ENVIRONMENT RISK OF ABANDONED PLANTS

(71) Applicant: Environmental Protection Administration, R.O.C. (Taiwan), Taipei City (TW)

(72) Inventors: Hung-Teh TSAI, Taipei City (TW); Kai-Hsing YANG, Taipei City (TW); Shen-De CHEN, Taipei City (TW); Yung-Kai KUAN, Taipei City (TW); Tung-Ching SUN, Taipei City (TW)

(73) Assignee: Environmental Protection Administration, R.O.C. (Taiwan), Taipei City (TW)

(21) Appl. No.: 14/540,063

(22) Filed: Nov. 13, 2014

(30) Foreign Application Priority Data
Aug. 5, 2014 (TW) 103126785

(51) Int. Cl. G01N 33/18; G06Q 10/06; G06F 17/30; G01D 1/02

(52) U.S. Cl. CPC G01N 33/18; G01D 1/02; G06Q 10/0635; G06F 17/30595

(57) ABSTRACT

An environment risk sorting method includes preparing a plant environment database for storing several pieces of environment risk data for abandoned plants; using a first risk evaluation module, based on the environment risk data, to generate a first risk evaluation result for each abandoned plant; using the first risk evaluation result to generate an environmental site evaluation list, which in turn, generates an environment site evaluation data of each abandoned plant and updated environmental risk data; using a second risk evaluation module, based on the environment site evaluation data of each abandoned plant and the updated environmental risk data, to generate a second risk evaluation result for each abandoned plant; and using the second risk evaluation result for each abandoned plant to generate an investigation list as a basis for follow-up investigation.

### CROSS-REFERENCE TO RELATED APPLICATION

This application claims the priority of Taiwanese patent application No. 103126785, filed on Aug. 5, 2014, which is incorporated herewith by reference.

### BACKGROUND OF THE INVENTION

1. Field of the Invention

The present invention relates generally to an environment risk sorting method, and more particularly an environment risk sorting method for controlling abandoned plants which cause high environmental pollution.

2. The Prior Arts

Industrial pollution is one major cause for underground water pollution, since wastes or pollutants are directly or indirectly discharged into the ground without adequate treatment to remove harmful compounds. Some abandoned plants cause high environment risk to the ground upon which the plants are built prior to shutdown or abandonment. The currently running plants need more attention since they constantly generate environment risk if no proper environment safety measurement is taken during the running period, and the land left by those abandoned plants may be contaminated. So that an environment risk sorting method is required to effectively sorting each of the abandoned plants as the basis for follow-up investigation for controlling the abandoned plants.

A conventional method of sorting abandoned plants causing high environment risk includes fetching the recorded basic information stored within an abandoned plant and evaluating the basic information so as to generate a risk evaluation result for each abandoned plant, which in turn, produces an environment site evaluation list. Since the environment site evaluation list includes names of abandoned plants and location sites, it is taken as the basis for follow-up investigation and management of the location sites. It is noted the earlier basic information of the abandoned plants are recorded not in properly arranged system or lacking such as environment risk level and hence will not be a perfect one owing to exclusion of the recent environment site evaluation information. The environment risk level of each abandoned plant is defined by the recorded information of the respective abandoned plant, thereby causing difficulty in obtaining the on-spot environment risk evaluation of the abandoned plant.

Therefore, the conventional method of environment risk sorting cannot handle a tremendously large amount of record information and incomplete information leads to waste of human labor, time, and cost, hence the management of material, needs to be upgraded urgently.

### SUMMARY OF THE INVENTION

The object of the present invention is to provide an environment risk sorting method that can eliminate the above-mentioned drawbacks resulted from the use of the conventional environment risk sorting method and that can update a plant environment database so as generate on-spot data of each abandoned plant, thereby obtaining an environment risk evaluation result substantially similar to the present condition so as to facilitate the follow-up management and investigation.

An environment risk sorting method of the present invention for controlling abandoned plants which cause high environmental pollution, includes: a preliminary step: preparing a plant environment database for storing several pieces of environment risk data for each abandoned plant, wherein the environment risk data includes pollution potential factor data and environment factor data; a first risk evaluation step: using a first risk evaluation module, based on the several pieces of environment risk data, to generate a first risk evaluation result for each abandoned plant, wherein the first risk evaluation result includes a first risk evaluation value (T₁); and a first risk level defined based on the first risk evaluation value (T₁); an environmental site evaluation step: using the first risk evaluation result to generate an environmental site evaluation list, which in turn, generates an environment site evaluation data of each abandoned plant and updated environmental risk data; a second risk evaluation step: using a second risk evaluation module, based on the environment site evaluation data of each abandoned plant and the updated environmental risk data, to generate a second risk evaluation result for each abandoned plant, wherein the second risk evaluation result includes a second risk evaluation value (T₂) and a second risk level defined based on the second risk evaluation value (T₂); and a risk managing step: using the second risk evaluation result for each abandoned plant to generate an investigation list as a basis for follow-up investigation.

Preferably, the preliminary step further includes a conditional sorting procedure that consists of plant site area of the environment risk data, plant location and storeys of each abandoned plant, and classification of abandoned plants into different groups based on usage change of each abandoned plant.

Preferably, in one embodiment of the present invention, the first risk evaluation value (T₁) is equivalent to a total sum of a first underground water environment risk factor (S_{gw,1}) and a first soil environment risk factor (S_{soil, 1}) and is multiplied by a weighting factor F. The first underground water environment risk factor (S_{gw, 1}) is computed from root mean square (RMS) of a first underground water pollution factor (P_{gw, 1}), an underground environmental vulnerability factor (C_{gw}) and an underground water pollution receptor factor (D_{gw}), wherein, the first underground water pollution factor (P_{gw, 1}) is computed from accumulated value of the environment risk data. The first soil environment risk factor (S_{soil,1 1}) is computed from root mean square (RMS) of a first soil pollution potential factor (P_{soil, 1}), a soil environmental vulnerability factor (Cₛₒᵢₗ) and a soil pollution receptor factor (Dₛₒᵢₗ), wherein, the first soil pollution potential factor (P_{soil, 1}) is computed from accumulated value of the environment risk data.

Preferably, the environment risk data includes a pollution potential factor (P), which consists of the following factors: registered plant site area (A₁), plant year of running (A₂), former record for atmosphere, water, waste and poisonous substances (A₃), number of owner transfer (A₄), ground water pollution rate (A_{5gw}), soil pollution rate (A₅ₛₒᵢₗ), amount of discharged polluted water into the ground (B_{gw}), amount of soil pollution (B ₛₒᵢₗ) and human toxicity pollution potential (HTP), which includes human toxicity groundwater pollution (HTP_{gw}) and human toxicity potential soil pollution (HTPₛₒᵢₗ).

Preferably, the amount of discharged polluted water into the ground (B_{gw}) is the total units of polluted water discharged within a plurality of designated periods. The amount of soil pollution (Bₛₒᵢₗ) is the total units of polluted soil caused within a plurality of designated periods.

Preferably, the environment site evaluation list includes an environment site evaluation list of high risk level including advising to conduct on-spot environment site evaluation for each abandoned plant; an environment site evaluation list of middle high risk level including determining environment site evaluation procedure of abandoned plants based on the pollution potential factor; an environment site evaluation list of middle risk level including further soil observation and managing of abandoned plants; and an environment site evaluation list of low risk level including no obvious public environment risk, no need of follow-up investigation.

Preferably, the second risk evaluation value (T₂) is equivalent to a total sum of a second groundwater environment risk factor (S_{gw, 2}) and a second soil environment risk factor (S_{soil, 2}) and is multiplied by a weighting factor (F). The second groundwater environment risk factor (S_{gw, 2}) is computed from root mean square (RMS) of a second underground water pollution potential factor (P_{gw, 2}), an underground environmental vulnerability factor (C_{gw}) and an underground water pollution receptor factor (D_{gw}), wherein the second underground water pollution potential factor (P_{gw, 2}) is computed from the environment risk data and the environment site evaluation data.

Preferably, the environment site evaluation data includes the following factors: plant running quality factor (I₁), plant facilities factor (I₂), history of plant relocation factor (I₃), previous environmental spill or accident factor (I₄), pollution potential factor (I₅) and change in land or land quality inspection rating factor (I₆).

Preferably, the investigation list includes an investigation list of high environment risk level including advising to conduct on-spot investigation of abandoned plants; and an investigation list of middle high environment risk level including advising to conduct further investigation of abandoned plants.

The second risk evaluation result includes a second risk evaluation value, from which, a second risk level is defined, wherein, the investigation list is defined based on the second risk level so as to facilitate the follow-up management and investigation, which in turn, economizes human labor and time.

### BRIEF DESCRIPTION OF THE DRAWINGS

The present invention will be apparent to those skilled in the art by reading the following detailed description of a preferred embodiment thereof, with reference to the attached drawings, in which:

FIG. 1 illustrates a block diagram including the steps of a method of the present invention for sorting environment risk in order to control abandoned plants which cause environmental pollution.

### DETAILED DESCRIPTION OF THE PREFERRED EMBODIMENT

The accompanying drawings are included to provide a further understanding of the invention, and are incorporated in and constitute a part of this specification. The drawings illustrate embodiments of the invention and, together with the description, serve to explain the principles of the invention.

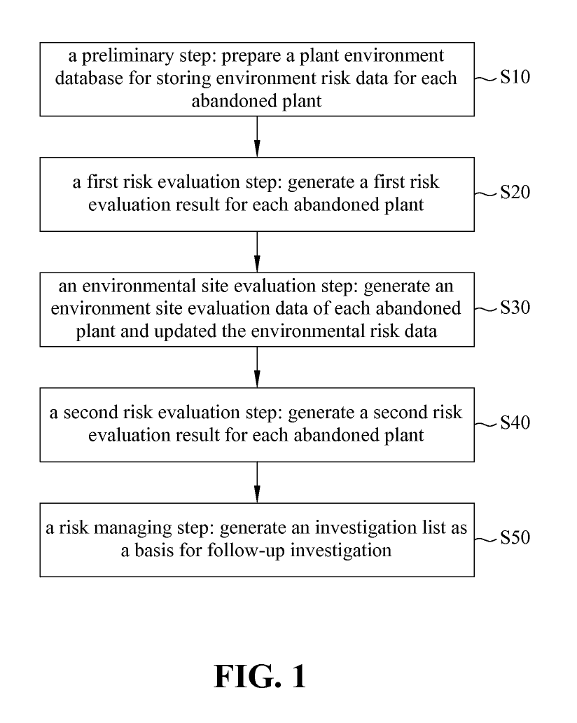

FIG. 1 illustrates a block diagram including the steps of a method of the present invention for sorting environment risk in order to control abandoned plants which cause environmental pollution. As illustrated, in a preliminary step S10: preparing a plant environment database for storing several pieces of environment risk data for each abandoned plant, wherein the environment risk data preferably include pollution potential factor data and environment factor data. In one embodiment of the present invention, the pollution potential factor data consists of the following factors registered plant site area (A₁); plant year of running (A₂); former record for atmosphere, water, waste and poisonous substances (A₃); number of owner transfer (A₄); ground water pollution rate (A_{5gw}); soil pollution rate (A₅ₛₒᵢₗ); amount of discharged polluted water into the ground (B_{gw}); amount of soil pollution (Bₛₒᵢₗ); and human toxicity pollution potential (HTP), which includes human toxicity groundwater pollution (HTP_{gw}) and human toxicity potential soil pollution (HTPₛₒᵢₗ). The environment factor data preferably include infiltration amount factor (C₁); topography factor (C₂); soil medium factor (C₃); ventilation layer medium factor (C₄); hydraulic conductivity coefficient factor (C₅); groundwater depth factor (C₆); aquifer medium factor (C₇); population density factor (D₁), soil /sediment contact risk factor (D₂) and groundwater contact risk factor (D₃).

In a first risk evaluation step S20: using a first risk evaluation module to generate a first risk evaluation result for each abandoned plant, wherein the first risk evaluation result includes a first risk evaluation value (T₁); and a first risk level defined based on the first risk evaluation value (T₁). In one embodiment, the first risk evaluation module utilizes the following equation (1) to generate the first risk evaluation value (T₁). In this embodiment, the first risk evaluation value (T₁) is computed from the following equation 1:

T₁=(S_{gw,1}+S_{soil,1})×F (1)

In the above equation, S_{gw, 1} is a first underground water environment risk factor; S_{soil,1} is a first soil environment risk factor; and F is a weighting factor. Assuming the weighting factor F=1.5, the first underground water environment risk factor S_{gw, 1} and the first soil environment risk factor S_{soil, 1} can be computed respectively from the equations (2) and (3);

S_{gw,1}=\sqrt{(P_{gw,1}²+C_{gw}²+D_{gw}²)/3} (2)

S_{soil,1}=\sqrt{(P_{soil,1}²+Cₛₒᵢₗ²)/3} (3)

In the above equation, P_{gw, 1} and P_{soil, 1} respectively represent the first underground water and potential soil pollution factors; C_{gw} and Cₛₒᵢₗ respectively stand for underground water and soil environment vulnerability factor; and D_{gw} and Dₛₒᵢₗ respectively represent underground water and soil pollution receptor factor. According to the environment risk data, the first underground water (P_{gw, 1}) and the first potential soil pollution factor (P_{soil, 1}) from the equations (2) and (3) can be computed from the following equations (4) and (5). However, owing to different running operation of the plants and time differences, the total sum for the pollution potential value of each abandoned plant is:

P_{gw, 1}=Σ/(A₁×A₂×B_{gw}×I₁×(1+A₃)×I₂) (1+A₄×I₃+I₄)×I₅×(1+A_{5gw})×HTP_{gw}]×I₆ (4)

P_{soil,1}=Σ/(A₁×A₂×Bₛₒᵢₗ×I₁×(1+A₃)×I₂) (1+A₄×I₃+I₄)×I₅×(1+A₅ₛₒᵢₗ)×HTPₛₒᵢₗ]×I₆ (5)

The underground water and soil environment vulnerability factors C_{gw} and Cₛₒᵢₗ of the equations (2) and (3) can be computed from the following equations (6) and (7);

C_{gw}=C₁+C₅+C₆+C₇ (6)

Cₛₒᵢₗ=C₂+C₃+C₄ (7)

The underground water factor (D_{gw}) and the soil pollution receptor factor (Dₛₒᵢₗ) of equations (2) and (3) can be computed from the following equations (8) and (9);

D_{gw}=D₁+D₃ (8)

Dₛₒᵢₗ=D₁+D₂ (9)

The corresponding value for the previous air pollution, water pollution, waste, poisonous chemical record factors (A3) are shown in table 1, the corresponding value of plant transfer time factor (A4), if transfer of the plant ownership take place once 1, if there is no transfer of plant ownership 0; the corresponding value for infiltration amount factor (C1), topographical factor (C2), soil medium factor (C3) and ventilation layer medium factor (C4) are shown in table 2; the corresponding value for hydraulic conductivity factor (C5), groundwater depth factor (C6) and aquifer medium factor (C7) are shown in table 3; and the corresponding value for population density factor (D1), soil/sediment contact risk factors (D2), and groundwater risk contact factors (D3) are shown respectively in tables 4--6. Amount of discharged polluted water into the ground (Bgw), amount of soil pollution (B soil), human toxicity pollution potential (HTP), previous environmental spill or accident factor (I4) (A5) are shown respectively in tables 7 and 8. If there is one time occurrence of environmental spill or accident, the accident factor (I4) is increased 1 while if there is no occurrence of environmental spill or accident, the accident factor (I4) is equivalent to 0.

TABLE 1
the corresponding potential factor values for
the previous air pollution, water pollution,
waste, poisonous chemical record factors

| Previous air pollution, water | A₃ corresponding to |
|---|---|
| pollution, waste, poisonous | pollution potential |
| chemical record factors | factor |
| No record | 0 |
| The submitted previous air pollution, | 0.5 |
| water pollution, waste, poisonous | |
| chemical record factors | |
| The previous environment water | 1 |
| pollution record | |

TABLE 2
C₁~C₄ transfer factor of the corresponding transfer factor value

| | | (C₂) | | | | | |
|---|---|---|---|---|---|---|---|
| (C₁) | | topo- | | | | (C₄) | |
| infil- | | graphical | | (C₃) | | ventilation | |
| tration | | incli- | | soil | | layer | |
| amount | val- | nation | val- | medium | Val- | medium | Val- |
| (cm/year) | ue | (%) | ue | and pH | ue | factor | ue |
| 0~2.5 | 0.8 | 18~99 | 0.6 | >10 | 1.2 | basalt | 1.2 |
| 2.5~5 | 1.6 | 15~18 | 1.2 | 9 | 2.4 | dolomite | 2.4 |
| 5~6.7 | 2.4 | 12~15 | 1.8 | 8 | 3.6 | marble | 3.6 |
| 6.7~8.3 | 3.2 | 10~12 | 2.4 | 7 | 4.8 | limestone | 4.8 |
| 8.3~10 | 4 | 6~10 | 3 | 6 | 6 | coal | 6 |
| 10~14 | 4.8 | 5~6 | 3.6 | 5 | 7.2 | quartz | 7.2 |
| | | | | | | schist | |
| 14~17 | 5.6 | 4~5 | 4.2 | 4 | 8.4 | marl | 8.4 |
| 17~25 | 6.4 | 3~4 | 4.8 | 3 | 9.6 | claystone | 9.6 |
| >25 | 8 | 2~3 | 5.4 | 2 | 10.8 | silty clay | 10.8 |
| — | — | 0~2 | 6 | 1 | 12 | clay | 12 |

TABLE 3
C₅~C₇ transfer factor of the corresponding transfer factor value

| (C₅) | | (C₆) | | (C₇) | |
|---|---|---|---|---|---|
| hydraulic | | groundwater | | aquifer | |
| conductivity | | depth | | medium | |
| factor (m/day) | Value | factor (m) | Value | factor | Value |
| <4 | 0.6 | 0~1.5 | 10 | Quartzite, granite | 0.6 |
| 4~8 | 1.2 | 1.5~3.5 | 9 | quartz schist, | 1.2 |
| | | | | red clay | |
| 8~12 | 1.8 | 3.5~4.5 | 8 | schist, shale | 1.8 |
| 12~20 | 2.4 | 4.5~6.75 | 7 | limestone, | 2.4 |
| | | | | ansun rock | |
| 20~28 | 3 | 6.75~9 | 6 | silty sand, | 3 |
| | | | | silty clay | |
| 28~34 | 3.6 | 9~12 | 5 | marble, coal | 3.6 |
| 34~40 | 4.2 | 12~15 | 4 | volcanic rock, | 4.2 |
| | | | | sandy gravel | |
| 40~60 | 4.8 | 15~22.5 | 3 | domolite | 4.8 |
| 60~80 | 5.4 | 22.5~30 | 2 | basalt | 5.4 |
| >80 | 6 | >30 | 1 | boulder | 6 |

TABLE 4
population density factor and
the corresponding factor value

| (D₁) density (person/km2) | value |
|---|---|
| ≦500 | 1 |
| 500~1000 | 2 |
| 1000~5000 | 4 |
| 5000~12000 | 6 |
| 12000~47000 | 8 |
| ≧47000 | 10 |

TABLE 5
soil/sediment contact risk factors and the corresponding factor value

| Soil | | (D₂) |
|---|---|---|
| implementation | | soil/sediment |
| level | Receptor | contact risk factors |
| Agriculture | Farmer:resident | 20 |
| Aquaculture | aquaculture industry: | 16 |
| | resident | |
| Forest | Visitor | 2 |
| Traffic | Visitor | 0 |
| Reservoir | Farmer:resident | 16 |
| Groundwater well | Farmer:resident | 0 |
| Water | staff: visitor | 4 |
| conservancy | | |
| Residential house | resident, children | 6 |
| Business | staff: resident | 4 |
| Culture & | Children:resident | 6 |
| education | | |
| Building | Staff:resident | 4 |
| Environment | Staff:resident | 4 |
| Industry | Staff:resident | 4 |
| Medical treatment | Staff:resident | 4 |
| Public | Staff:resident | 4 |
| Recreation | Visitor | 2 |

TABLE 6
groundwater risk contact potential factors and
the corresponding factor value

| Soil | | (D₃) groundwater |
|---|---|---|
| implementation | | risk contact |
| classification | Receptor | potential factor |
| Agriculture | Farmer:resident | 6 |
| Aquaculture | Aquaculture | 6 |
| Industry: resident | | |
| Forest | Visitor | 2 |
| Traffic | Visitor | 0 |
| Reservoir | Farmer:resident | 6 |
| Groundwater well | Farmer:resident | 20 |
| Water | Staff:visitor | 6 |
| conservancy | | |
| Residential | Resident:children | 20 |
| housing | | |
| Business | Staff:resident | 6 |
| Culture and | Children:Resident | 6 |
| Education | | |
| Building | Staff:resident | 6 |
| Environment | Staff:resident | 6 |
| Industry | Staff:resident | 6 |
| Medical treatment | Staff:resident | 6 |
| Public | Staff:resident | 6 |
| Recreation | Visitor | 2 |

TABLE 7
amount of polluted water discharged into the ground and soil pollution caused thereby

| | | Discharge of polluted water | | Soil pollution caused | |
|---|---|---|---|---|---|
| | | into the ground (in unit) | | thereby (in unit) | |
| Category | | Before yr 94 | After yr 94 | Before yr 94 | After yr 94 |
| Code | Type of industry | B_{gw94} | B_{gw94} | Bₛₒᵢₗ₉₄ | Bₛₒᵢₗ₉₄ |
| 13 | Fur and Leather Product | 1.49E−04 | 5.77E−05 | 3.11E−04 | 6.68E−05 |
| | Manufacturing | | | | |
| 14 | Wood and bamboo | 1.18E−06 | 1.00E−06 | 1.26E−05 | 3.98E−06 |
| | manufacturing | | | | |
| 17 | Petroleum and Coal Products | 9.10E−06 | 2.95E−05 | 2.39E−05 | 4.04E−05 |
| 1810 | Basic Chemical Industries | 5.07E−05 | 1.24E−04 | 6.85E−05 | 9.89E−05 |
| 1820 | Petrochemicals | 6.58E−05 | 2.62E−04 | 8.88E−05 | 2.08E−04 |
| 1830 | Fertilizer Manufacturing | 3.06E−05 | 3.81E−05 | 2.55E−05 | 3.18E−05 |
| 1841 | Synthetic resin industry | 2.51E−05 | 5.28E−05 | 8.61E−05 | 1.81E−04 |
| 1842 | Synthetic Rubber | 9.65E−05 | 2.09E−04 | 1.30E−04 | 1.66E−04 |
| | Manufacturing | | | | |
| 1850 | Rayon | 7.11E−04 | 5.89E−04 | 1.66E−04 | 1.37E−04 |
| | manufacturing | | | | |
| 1910 | Pesticides and Herbicides | 5.06E−07 | 8.33E−07 | 7.26E−06 | 1.20E−05 |
| | manufacturing | | | | |
| 1920 | Paint, dye and pigment | 1.08E−04 | 1.01E−04 | 1.45E−04 | 8.02E−05 |
| | manufacturing | | | | |
| 22 | Plastic Products Manufacturing | 1.54E−05 | 1.55E−05 | 1.19E−04 | 3.41E−05 |
| 24 | Basic metalindustries | 7.88E−04 | 1.10E−03 | 9.78E−04 | 1.32E−03 |
| 2543 | Metal Heat Treatment | 2.52E−05 | 3.36E−05 | 6.66E−05 | 4.58E−05 |
| | Industry | | | | |
| 2544 | Metal surface treatment | 3.59E−05 | 2.51E−05 | 9.50E−05 | 3.42E−05 |
| | industry | | | | |
| 26 | Electronic Components | 2.22E−05 | 3.46E−05 | 5.48E−05 | 4.05E−05 |
| | Manufacturing | | | | |
| 27 | Computer communications | 3.63E−05 | 4.38E−05 | 8.94E−05 | 5.13E−05 |
| | and audio-visual and | | | | |
| | electronic products | | | | |
| 28 | Power Supplies and | 2.97E−05 | 2.42E−05 | 1.12E−04 | 3.88E−05 |
| | Equipment Manufacturing | | | | |
| 381 | Waste collection | 2.09E−03 | 8.27E−04 | 2.04E−03 | 8.14E−04 |
| 382 | Waste Treatment Industry | 8.73E−04 | 4.09E−04 | 8.48E−04 | 4.03E−04 |

TABLE 8
potential toxicity weights caused by each industry

| Category Code | Type of industry | HTP_{gw} | HTPₛₒᵢₗ |
|---|---|---|---|
| 13 | Fur and Leather Product | 3.3E+01 | 4.2E−02 |
| | Manufacturing | | |
| 14 | Wood and bamboo manufacturing | 1.5E+02 | 2.9E−01 |
| 17 | Petroleum and Coal Products | 3.9E+01 | 2.2E−01 |
| 1810 | Basic Chemical Industries | 2.6E+02 | 3.8E−01 |
| 1820 | Petrochemicals | 3.5E+02 | 3.1E−01 |
| 1830 | Fertilizer Manufacturing | 1.1E−01 | 7.2E−03 |
| 1841 | Synthetic resin industry | 1.0E+02 | 1.6E−01 |
| 1842 | Synthetic Rubber Manufacturing | 2.1E+02 | 3.1E−01 |
| 1850 | Rayon manufacturing | 1.8E+00 | 2.1E−02 |
| 1910 | Pesticides and Herbicides | 1.2E+03 | 4.5E−01 |
| | manufacturing | | |
| 1920 | Paint, dye and pigment | 2.1E+02 | 3.1E−01 |
| | manufacturing | | |
| 22 | Plastic Products Manufacturing | 1.5E+02 | 3.0E−01 |
| 24 | Basic metal industries | 1.5E+02 | 3.0E−01 |
| 2543 | Metal Heat Treatment Industry | 1.5E+02 | 2.9E−01 |
| 2544 | Metal surface treatment industry | 1.5E+02 | 2.9E−01 |
| 26 | Electronic Components | 5.3E+01 | 2.8E−01 |
| | Manufacturing | | |
| 27 | Computer communications and | 5.3E+01 | 2.8E−01 |
| | audio-visual and electronic | | |
| | products | | |
| 28 | Power Supplies and Equipment | 1.5E+02 | 3.1E−01 |
| | Manufacturing | | |
| 381 | Waste collection | 1.3E+03 | 6.1E−01 |
| 382 | Waste Treatment Industry | 1.3E+03 | 6.1E−01 |

The first risk evaluation value (T₁) of the abandoned plants, and the accumulated of first risk level are shown in table 9. The high risk level and scope is 90% greater than the accumulated first risk evaluation value, i.e., the first risk evaluation value (T₁) ranges 60-100; the middle high risk level is 50%-90 greater than the accumulated first risk evaluation value, i.e., the first risk evaluation value (T₁) ranges 40-59, the middle risk level is greater than 10-50% of the accumulated first risk evaluation value, i.e., the first risk evaluation value (T₁) ranges 30-39 while the low risk level is 10% smaller than the accumulated first risk evaluation value, i.e., the first risk evaluation value (T₁) ranges 0-29.

TABLE 9
The first risk evaluation value, accumulated
risk and risk level of abandoned plants

| | Accumulated | First risk |
|---|---|---|
| First risk level | risk (%) | evaluation value |
| Low risk level | 0 | 20.86 |
| Middle | 10 | 32.32 |
| risk | 20 | 34.21 |
| level | 30 | 36.19 |
| | 40 | 37.94 |
| Middle | 50 | 41.53 |
| high risk | 60 | 50.23 |
| level | 70 | 53.37 |
| | 80 | 56.41 |
| High risk | 90 | 59.55 |
| level | 100 | 78.13 |

In the environment site evaluation step S30, the investigator fetches the environment site evaluation data and the updated environment risk data. Firstly, the environment site evaluation list is defined based on the first risk level and the first risk evaluation value (T₁). That is if the first risk evaluation value (T₁) ranges 60-100, an environment site evaluation list of high risk level is defined. If the first risk evaluation value (T₁) ranges 40-59, an environment site evaluation list of middle high risk level is defined. If the first risk evaluation value (T₁) ranges 30-39, an environment site evaluation list of middle risk level is defined. If the first risk evaluation value (T₁) ranges 0-29, an environment site evaluation list of low risk level is defined. Afterward, the abandoned plants in the environment site evaluation list of the high or middle risk level are further classified again so as to update the environment site evaluation data and the environment risk data, wherein the environment site evaluation data includes the following factors: plant running quality factor (I₁), plant facilities factor (I₂), history of plant relocation factor (I₃), previous environmental spill or accident factor (I₄), pollution potential factor (I₅) and change in land or land quality inspection rating factor (I₆).

In the second risk evaluation step S40: the investigator fetches the updated environment risk data and the environment site evaluation data via the second risk evaluation module as in the first risk evaluation step S30 to compute out the second risk evaluation result for each abandoned plant, wherein the second risk evaluation result in fact is the second risk evaluation value (T₂), from which the second risk level is defined. The second risk evaluation value (T₂) can be computed from the following equation 10:

T₂−(S_{gw,2}+S_{soil,2})×F (10)

In the above equation, S_{gw, 2} is a second groundwater environment risk factor; S_{soil, 2} is the second soil environment risk factor; F is the weighting factor, in one embodiment it is assumed as 1.5. The second groundwater environment risk factor S_{gw, 2} and the second soil environment risk factor S_{soil, 2} of the above equation (10) can be computed from the following equations (11) and (12) respectively:

S_{gw,2}=\sqrt{(P_{gw,2}²+C_{gw}²+D_{gw}²)/3} (11)

S_{soil,2}=\sqrt{(P_{soil,2}²+Cₛₒᵢₗ²+Dₛₒᵢₗ²)/3} (12)

In the above equation, P_{gw, 2} and P_{soil, 2} respectively represent the second ground water and the second soil pollution potential factor; C_{gw} and Cₛₒᵢₗ respectively represent ground water and soil environment vulnerability factor; D_{gw} and Dₛₒᵢₗ respectively represent ground water and soil pollution receptor factors. The second ground water P_{gw, 2} and the second soil pollution potential factor P_{soil, 2} of the above equations (11) and (12) are based on the updated environment risk data and the environment site evaluation data and can be computed from the following equations (13) and (14), wherein owing to different running operation of the plants and time differences, the total sum for the pollution potential value of each abandoned plant is:

P_{gw, 2}=Σ[(A₁×A₂×B_{gw}×I₁×(1+A₃)×I₂) (1+A₄×I₃+I₄)×I₅×(1+A_{5gw})×HTP_{gw}]×I₆ (13)

P_{soil, 2}=Σ[(A₁×A₂×Bₛₒᵢₗ×I₁×(1+A₃)×I₂) (1+A₄×I₃+I₄)×I₅×(1+A₅ₛₒᵢₗ)×HTPₛₒᵢₗ]×I₆ (14)

The evaluated ways of the updated environment risk data is the same as the previous environment risk data. The environment site evaluation factor data includes the following factors: plant running quality factor (I₁), plant facilities factor (I₂), history of plant relocation factor (I₃), previous environmental spill or accident factor (I₄), pollution potential factor (I₅) and change in land or land quality inspection rating factor (I₆) obtained from Table 10. An important aspect to note is that for the common knowledge in this technical field, Table 10 clearly shows the sorting level and recycling of the value of environment site evaluation is likely happened. Preferably, the value of environment site evaluation data of the present invention is determined by professional persons, who possess the following qualifications (1) professional technicians possessing environment engineering, applied geology, geotechnical engineering practice license; (2) persons having more than three years, soil or groundwater pollution investigation after completing at least Master Degree from public or private university or independent college recognized by the Ministry of Education or foreign university in engineering or site assessment of relevant work experience; and (3) persons having more than five years in soil or groundwater pollution investigation after graduating in the engineering, agriculture, medicine field from public or private university recognized by the Ministry of Education or foreign university or relevant work experience. More preferably, the previously mentioned persons should have been trained through the government agency in charge of environmental protection, and have passed or completed the test concerning environmental protection.

TABLE 10
factor correction table for sorting abandoned plant
through the second risk evaluation

| Name of | Amended | | | |
|---|---|---|---|---|
| factor | code | Description | | Difference |
| Operation | I₁ | To further control operation quality of plants via | | New |
| quality of | | the environment site evaluation data, the | | |
| plant | | evaluation value is set in advance 1: | | |
| | | I₁ | Highlight | |
| | | 1 | No specific plant data except the | |
| | | | speculated pollution generating | |
| | | | substances used by the plant owner | |
| | | | or list of some control of pollutants | |
| | | 0.8 | The controlled pollutants include | |
| | | | such as dioxins, PCBs, pesticides, | |
| | | | chlorinated organics, which are | |
| | | | difficult to break down according to | |
| | | | EPA method of soil pollution in the | |
| | | | environment | |
| | | 0.6 | controlled pollutants include such | |
| | | | as heavy metals, volatile organic | |
| | | | solvents, oil of non-pollution of soil | |
| | | | according to EPA regulated law | |
| | | 0.4 | Plant operation includes pollutants, | |
| | | | like volatile organic solvents, | |
| | | | semi-volatile organic solvents, | |
| | | | toxic chemicals, and other | |
| | | | environmental hormones which | |
| | | | are excluded from EPA regulated | |
| | | | law | |
| | | 0.1 | No pollutants found during | |
| | | | operation of plant | |
| Process | I₂ | To further control process devices of plants via | | New |
| devices | | the environment site evaluation data, the | | |
| rating | | evaluation value is set in advance 1: | | |
| | | I₂ | Highlight | |
| | | 2.0 | Process devices include wet | |
| | | | operation, like wastewater treatment | |
| | | | facilities, underground storage tanks, | |
| | | | discharge ditches or pipes, sumps or | |
| | | | wells | |
| | | 1.6 | Process devices includes ground | |
| | | | storage tank, tank and other facilities | |
| | | | for loading liquid, raw materials, | |
| | | | semi-finished or finished products | |
| | | 1.4 | Process devices generates dust, | |
| | | | slag, bottom ash and other waste | |
| | | | containing heavy metals | |
| | | 0.5 | Process devices use organic solvents | |
| | | | for cleaning and wiping, dilution and | |
| | | | coating, but no wastewater generated | |
| | | 0.1 | No significant pollution potential, | |
| | | | such as plastic injection, wood | |
| | | | cutting, assembling parts found | |
| history of | I₃ | To further understand history of plant relocation | | New |
| plant | | via the environment site evaluation data, the | | |
| relocation | | evaluation value is set in advance 1: | | |
| | | I₃ | Highlight | |
| | | 1 | No relocation found | |
| | | 0 | Located at its initial spot, no | |
| | | | relocation recorded | |
| Occurrence | I₄ | To further understand safety of working | | New |
| of pollution | | environment via the environment site evaluation | | |
| or accident | | data, the evaluation value is set in advance 0: | | |
| | | I₄ | Highlight | |
| | | 1 | Each leak of pollutant, outbreak of | |
| | | | fire or accident in short time results | |
| | | | in 1 score | |
| | | 0 | No record of leak of pollutant, | |
| | | | outbreak of fire or any accident | |
| Potential | I₅ | To further understand the status of plant pollution | | New |
| pollution | | via the environment site evaluation data, the | | |
| rating | | evaluation value is set in advance 1: | | |
| | | I₅ | Highlight | |
| | | 2.0 | On-spot inspection finds dumping, | |
| | | | broken or damaged tank, tank or | |
| | | | sump; or dust collection, slag, and | |
| | | | other spilling, sediment spreading | |
| | | | circumstances | |
| | | 1.6 | On-spot inspection finds bare | |
| | | | ground, abnormal color, | |
| | | | projections, and piles of unknown | |
| | | | substances or soil | |
| | | 1.4 | cracks in cement floor, patching on | |
| | | | floor, water pool or pond | |
| | | 0.5 | The initial plant is brick or iron | |
| | | | building, no demolition or | |
| | | | alteration records, no obvious | |
| | | | abnormalities | |
| | | 0.1 | The initial plant is reinforced | |
| | | | concrete buildings, no demolition | |
| | | | or alteration record, no significant | |
| | | | pollution found. | |
| soil or | I₆ | To further understand the soil quality of plant via | | New |
| land | | the environment site evaluation data, the | | |
| quality | | evaluation value is set in advance 1: | | |
| | | I₆ | Highlight | |
| | | 0.5 | Under environment evaluation by | |
| | | | authority concerned, no record of | |
| | | | soil or land pollution found in the | |
| | | | plant. | |
| | | 0.1 | Inspection of soil or land carried | |
| | | | out under regulations 8 and 9, no | |
| | | | record of soil or land pollution | |
| | | | found in the plant. | |
| | | 0.01 | The plant's location was a former | |
| | | | living community and hence | |
| | | | undergone land renovation | |

In the risk managing step S50, an investigation list of each abandoned plant is generated based on the second risk evaluation result as a basis for follow-up investigation. Firstly, the investigation list is defined based on the second risk level and the second risk evaluation value (T₂), that is a high risk investigation list is defined when the second risk evaluation value (T₂) ranges 60-100%, a middle high risk investigation list is defined when the second risk evaluation value (T₂) ranges 40-59%; a middle risk investigation list is defined when the second risk evaluation value (T₂) ranges 30-39% and a low risk investigation list is defined when the second risk evaluation value (T₂) ranges 0-29%. Afterward, based those investigation lists, the following Table 11 is drawn as a basis for follow-up investigation, wherein plant A . . . I indicates code for abandoned plant.

TABLE 11
investigation list and managing plan

| List | Abandoned plant | Follow-up managing plan |
|---|---|---|
| Low risk | Plant A | No obvious public environment |
| investigation list | | risk, no need of follow-up |
| | | investigation. |
| Middle risk | Plant D, F | No obvious public environment |
| investigation list | | risk, soil follow-up investigation is |
| | | needed. |
| Middle high risk | Plant B, C | Based on soil and groundwater |
| investigation list | | pollution potential factor value, |
| | | plant evaluation is required. |
| High risk | Plant E, G, H, I | Need on-spot site investigation |
| investigation list | | |

In one embodiment of the present invention, the preliminary step further includes a conditional sorting procedure that consists of plant site area of the environment risk data, plant location and storeys of each abandoned plant, and classification of abandoned plants into different groups based on usage change of each abandoned plant so as to serve as follow-up investigation for the specific group with lesser pollution, thereby avoiding the undesired investigation process. For instance, the abandoned plant concerns metal industry, having a surface area 1378 square meter, registered in 1995, abandoned in 2003, the registered address is No. XXX, 4F, X road and etc. According to the environmental risk data, the above abandoned plant is included in the middle high risk list based on the first risk evaluation result, a further investigation is needed. Since the abandoned plant is located at 4^{th} floor, there should not be any soil or water pollution potential data. Owing to the merit of conditional sorting procedure, the above mentioned plants can be re-classified into another groups, thereby avoiding the environment risk evaluation resulted from the first risk evaluation list.

In one embodiment of the present invention, under different environment protection laws of different periods, the amount of discharged polluted water into the ground (B_{gw}) and the amount of soil pollution (Bₛₒᵢₗ) caused thereby by a certain type industry are relatively high or low. Therefore, in order to avoid the mis-evaluation of the discharged pollutants calculated by average mean of the total years, the abandoned plants are classified into different groups based on discharged amount of polluted water into the ground (B_{gw}) and the amount of soil pollution (Bₛₒᵢₗ) according to the designated time and year. For instance, the person concerned wishes to investigate one particular plant abandoned before the year 2005, the amount of discharged polluted water into the ground (B_{gw, before 2005}, B_{gw, after 2005}) and the amount of soil pollution (B_{soil, before 2005}, B_{soil, after 2001}) caused by one specified industry before and after Jan. 1, 2005 are evaluated as the basis of the follow-up investigation so that he can evaluate the amount of polluted water discharged into the ground (B_{gw}) and the amount of soil pollution (Bₛₒᵢₗ) caused by that specified industry or plant abandoned after the year 2005.

The environment site evaluation step S30 includes fetching the on-spot environment risk data of each abandoned plant and since the environment risk data within the environment plant database is inconsistent with the on-sport environment risk data owing to incomplete record of the abandoned plant. Under this condition, the environment site evaluation step includes fetching the on-spot environment risk data of each abandoned plant and updating the environment risk data within the environment plant database, wherein the on-spot environment risk data includes the on-spot pollution potential factor data and the on-spot environment factor data. Preferably, the on-spot pollution potential factor data includes the on-spot plant area (A₁′), the on-spot plant running yea (A₂′), the previous air pollution, water pollution, waste, poisonous record factors (A₃′), the on-spot plant transfer time factor (A₄′), the on-spot potential ground water pollution (A_{5gw}) of a respective plant (A_{5gw}′), the on-spot potential soil pollution (A₅ₛₒᵢₗ′), the on-spot amount of discharged polluted water into the ground (B_{gw}′), the on-spot amount of soil pollution (Bₛₒᵢₗ′), the on-spot human toxicity potential groundwater pollution (HTP_{gw}′), and the on-spot human toxicity potential soil pollution (HTPₛₒᵢₗ′). The on-spot environment risk data preferably include the on-spot infiltration amount factor (C₁′), the on-spot topography factor (C₂′), the on-spot soil medium factor (C₃′), the on-spot ventilation layer medium factor (C₄′), the on-spot hydraulic conductivity coefficient factor (C₅′), the on-spot groundwater depth factor (C₆′), the on-spot aquifer medium factor (C₇′), the on-spot population density factor (D₁′) and the on-spot soil /sediment contact risk factor (D₂′).

As explained above, the following advantages are achieved. The investigator is capable of fetching the first environment risk evaluation list based on the conditional sorting procedure, thereby narrowing the scope of the environment site risk evaluation and thus obtaining the environment site evaluation list including the environment evaluation data and the updated environment risk data. Afterward, he is capable of fetching the second environment risk evaluation list based on the environment evaluation data and the updated environment risk data such that the second environment risk evaluation list is generally similar to the present condition of each abandoned plant, thereby facilitating the sorting environment risk for controlling abandoned plants.

Although the present invention has been described with reference to the preferred embodiments thereof, it is apparent to those skilled in the art that a variety of modifications and changes may be made without departing from the scope of the present invention which is intended to be defined by the appended claims.

1. A method for sorting environment risk for controlling abandoned plants which cause environmental pollution, comprising:
a preliminary step: preparing a plant environment database for storing several pieces of environment risk data for each abandoned plant;
a first risk evaluation step: using a first risk evaluation module, based on said several pieces of environment risk data, to generate a first risk evaluation result for each abandoned plant;
an environmental site evaluation step: using said first risk evaluation result to generate an environmental site evaluation list, which in turn, generates an environment site evaluation data of each abandoned plant and updated environmental risk data;
a second risk evaluation step: using a second risk evaluation module, based on said environment site evaluation data of each abandoned plant and said updated environmental risk data, to generate a second risk evaluation result for each abandoned plant; and
a risk managing step: using said second risk evaluation result for each abandoned plant to generate an investigation list as a basis for follow-up investigation.

2. The environment risk sorting method according to claim 1, wherein said preliminary step further includes a conditional sorting procedure that consists of plant site area of said environment risk data, plant location and storeys of each abandoned plant, and classification of abandoned plants into different groups based on usage change of each abandoned plant.

3. The environment risk sorting method according to claim 1, wherein said environment risk data includes pollution potential factor data and environment factor data.

4. The environment risk sorting method according to claim 1, wherein said first risk evaluation result includes
a first risk evaluation value (T₁); and
a first risk level defined based on said first risk evaluation value (T₁); wherein said environment site evaluation list is defined by said first risk level.

5. The environment risk sorting method according to claim 4, wherein said first risk evaluation value (T₁) is equivalent to a total sum of a first underground water environment risk factor (S_{gw, 1}) and a first soil environment risk factor (S_{soil, 1}) and is multiplied by a weighting factor F.

6. The environment risk sorting method according to claim 5, wherein said first underground water environment risk factor (S_{gw, 1}) is computed from root mean square (RMS) of a first underground water pollution factor (P_{gw, 1}), an underground environmental vulnerability factor (C_{gw}) and an underground water pollution receptor factor (D_{gw}), wherein, the first underground water pollution factor (P_{gw, 1}) is computed from accumulated value of said environment risk data.

7. The environment risk sorting method according to claim 5, wherein said first soil environment risk factor (S_{soil, 1}) is computed from root mean square (RMS) of a first soil pollution potential factor (P_{soil, 1}), a soil environmental vulnerability factor (Cₛₒᵢₗ) and a soil pollution receptor factor (Dₛₒᵢₗ)_{,} wherein, said first soil pollution potential factor (P_{soil, 1}) is computed from accumulated value of said environment risk data.

8. The environment risk sorting method according to claim 1, wherein said environment risk data includes a pollution potential factor data (P), which consists of the following factors:
registered plant site area (A₁);
plant year of running (A₂);
former record for atmosphere, water, waste and poisonous substances (A₃);
number of owner transfer (A₄);
ground water pollution rate (A_{5gw});
soil pollution rate (Aₛₒᵢₗ);
amount of discharged polluted water into the ground (B_{gw});
amount of soil pollution (Bₛₒᵢₗ); and
human toxicity pollution potential (HTP), which includes human toxicity groundwater pollution (HTP_{gw}) and human toxicity potential soil pollution (HTPₛₒᵢₗ).

9. The environment risk sorting method according to claim 8, wherein said amount of discharged polluted water into the ground (B_{gw}) is the total units of polluted water discharged within a plurality of designated periods.

10. The environment risk sorting method according to claim 8, wherein said amount of soil pollution (Bₛₒᵢₗ) is the total units of polluted soil caused within a plurality of designated periods.

11. The environment risk sorting method according to claim 1, wherein said environment site evaluation list includes
an environment site evaluation list of high risk level including advising to conduct on-spot environment site evaluation for each abandoned plant;
an environment site evaluation list of middle high risk level including determining environment site evaluation procedure of abandoned plants based on said pollution potential factor;
an environment site evaluation list of middle risk level including further soil observation and managing of abandoned plants; and
an environment site evaluation list of low risk level including no obvious public environment risk, no need of follow-up investigation

12. The environment risk sorting method according to claim 1, wherein said second risk evaluation result includes a second risk evaluation value (T₂); and
a second risk level defined based on said second risk evaluation value (T₂), wherein said investigation list is defined based on said second risk level.

13. The environment risk sorting method according to claim 12, wherein said second risk evaluation value (T₂) is equivalent to a total sum of a second groundwater environment risk factor (S_{gw, 2}) and a second soil environment risk factor (S_{soil, 2}) and is multiplied by a weighting factor (F).

14. The environment risk sorting method according to claim 13, wherein said second groundwater environment risk factor (S_{gw, 2}) is computed from root mean square (RMS) of a second underground water pollution potential factor (P_{gw, 2}), an underground environmental vulnerability factor (C_{gw}) and an underground water pollution receptor factor (D_{gw}), wherein said second underground water pollution potential factor (P_{gw, 2}) is computed from said environment risk data and said environment site evaluation data.

15. The environment risk sorting method according to claim 13, wherein said second soil environment risk factor (S_{soil, 2}) is computed from root mean square (RMS) of a second soil pollution potential factor (P_{soil, 2}), a soil environmental vulnerability factor (Cₛₒᵢₗ) and a soil pollution reception factor (Dₛₒᵢₗ), wherein said second soil pollution potential factor (P_{soil, 2}) is computed from said environment risk data and said environment site evaluation data.

16. The environment risk sorting method according to claim 12, wherein said environment site evaluation data includes the following factors:
plant running quality factor (I₁);
plant facilities factor (I₂);
history of plant relocation factor (I₃);
previous environmental spill or accident factor (I₄);
pollution potential factor (I₅); and
change in land or land quality inspection rating factor (I₆).

17. The environment risk sorting method according to claim 12, wherein said investigation list includes
an investigation list of high environment risk level including advising to conduct on-spot investigation of abandoned plants; and
an investigation list of middle high environment risk level including advising to conduct further investigation of abandoned plants.

18. The environment risk sorting method according to claim 1, wherein said environmental site evaluation step further includes fetching on-spot environment risk data of each abandoned plant based on said environment site evaluation list, updating data within said plant environment database based on said on-spot environment risk data so as to obtain updated environment risk data.

19. The environment risk sorting method according to claim 2, wherein said first risk evaluation result includes
a first risk evaluation value (T₁); and
a first risk level defined based on said first risk evaluation value (T₁); wherein said environment site evaluation list is defined by said first risk level.

20. The environment risk sorting method according to claim 19, wherein said first risk evaluation value (T₁) is equivalent to a total sum of a first underground water environment risk factor (S_{gw, 1}) and a first soil environment risk factor (S_{soil, 1}) and is multiplied by a weighting factor F.

21. The environment risk sorting method according to claim 20, wherein said first underground water environment risk factor (S_{gw, 1}) is computed from root mean square (RMS) of a first underground water pollution potential factor (P_{gw, 2}), an underground environmental vulnerability factor (C_{gw}) and a underground water pollution receptor factor (D_{gw}), wherein, the first underground water pollution factor (P_{gw, 1}) is computed from accumulated value of said environment risk data.

22. The environment risk sorting method according to claim 20, wherein said first soil environment risk (S_{soil, 1}) is computed from root mean square (RMS) of a first soil pollution potential factor (P_{soil, 1}), a soil environment vulnerability factor (Cₛₒᵢₗ) and a soil pollution reception factor (Dₛₒᵢₗ), wherein said first soil pollution potential factor (P_{soil, 1}) is computed from accumulated value of said environment risk data.

23. The enviroment risk sorting method according to claim 2, wherein said second risk evaluation result includes
a second risk evaluation value (T₂); and
a second risk level defined based on said second risk evaluation value (T₂), wherein said investigation list is defined based on said second risk level.

24. The enviroment risk sorting method according to claim 23, wherein said second risk evaluation (T₂) is equivalent to a total sum of a second groundwater environment risk factor (S_{gw,2}) and a second soil environment risk factor (S_{soil, 2}) and is multiplied by a weighting factor (F).

25. The enviroment risk sorting method according to claim 24, wherein said second groundwater environment risk factor (S_{gw, 2}) is computed from root mean square (RMS) of a second underground water pollution potential factor (P_{gw, 2}), an underground environmental vulnerability factor (C_{gw}) and a underground water pollution receptor factor (D_{gw}), wherein said second underground water pollution potential factor (P_{gw, 2}) is computed from said environment risk data and said environment site evaluation data.

26. The enviroment risk sorting method according to claim 24, wherein said second soil environment risk (S_{soil, 2}) is computed from root mean square (RMS) of a second soil pollution potential factor (P_{soil, 2}), a soil environment vulnerability factor (Cₛₒᵢₗ) and a soil pollution reception factor (Dₛₒᵢₗ), wherein said second soil pollution potential factor (P_{soil, 2}) is computed from said environment risk data and said environment site evaluation data.

27. The enviroment risk sorting method according to claim 23, wherein said environment site evaluation data includes the following factors:
plant running quality factor (I₁);
plant facilities factor (I₂);
history of plant relocation factor (I₃);
previous environmental spill or accident factor (I₄);
pollution potential factor (I₅); and
change in land or land quality inspection rating factor (I₆).

28. The enviroment risk sorting method according to claim 23, wherein said investigation list includes
an investigation list of high environment risk level including advising to conduct on-spot investigation of abandoned plants; and
an investigation list of middle high environment risk level including advising to conduct further investigation of abandoned plants.
